(19) United States
(12) Patent Application Publication
(10) Pub. No.: US 2024/0408056 A1
(43) Pub. Date: Dec. 12, 2024

(54) METHODS FOR REPAIRING NERVE DAMAGE USING PKC ACTIVATING THERAPEUTIC AGENTS

(71) Applicant: Synaptogenix, Inc., New York, NY (US)

(72) Inventor: Daniel L. ALKON, Chevy Chase, MD (US)

(21) Appl. No.: 18/740,967

(22) Filed: Jun. 12, 2024

Related U.S. Application Data
(60) Provisional application No. 63/472,425, filed on Jun. 12, 2023.

(51) Int. Cl. A61K 31/365; A61K 31/23

(52) U.S. Cl. CPC A61K 31/365; A61K 31/23

(57) ABSTRACT

A method for treating nerve damage in a subject in need thereof, the method comprising administering to the subject a pharmaceutically effective amount of a PKC activating therapeutic agent (e.g., bryostatin compound or analogue thereof or PUFA compound or derivative thereof) on a weekly dosage regimen to result in at least partial repair or regeneration of damaged nerves in the subject, wherein the weekly dosage regimen comprises a dosage of about 10 to about 60 μg/m² once a week for at least two weeks, wherein the at least two weeks of dosing are done consecutively or intermittently. The subject may have, for example, spinal cord injury, cochlear nerve damage, or nerve damage to direct peripheral nerves, such as in the extremities.

### CROSS REFERENCE TO RELATED APPLICATION

The present application claims benefit of U.S. Provisional Application No. 63/472,425, filed on Jun. 12, 2023, all of the contents of which are incorporated herein by reference.

### BACKGROUND

Conditions related to nerve or neuronal damage are well known, yet the options for treatment remain limited. Some of these conditions include spinal cord injury, cochlear nerve damage, and nerve damage to direct peripheral nerves, such as in the extremities. The nerve damage may, in some cases, be associated with diabetes or a physical injury. At present, the medical interventions for nerve damage are mainly reliant on managing the symptoms rather than treating the underlying cause, i.e., nerve damage. While helping to mitigate the symptoms is helpful for improving the patient's quality of life, many of the medical interventions require lifelong administration of medications and/or wearing of orthopedic devices. Moreover, oftentimes, the symptoms associated with nerve damage progress, which may warrant more aggressive ongoing therapies. Thus, to improve the patient's quality of life, there would be a significant benefit in a method that could more directly and effectively treat the nerve or neuronal damage itself.

### SUMMARY

The present disclosure is directed to methods for treating or preventing nerve damage, which may occur unassociated with another disorder or associated with another disorder or condition (e.g., diabetes, injury, infection, autoimmune disease, or tumor). The subject may have, for example, spinal cord injury, cochlear nerve damage, ALS, or nerve damage to direct peripheral nerves. The methods advantageously result in the repair or regeneration of damaged nerve tissue in the subject. Thus, the methods described herein more directly and effectively treat the nerve damage compared to the conventional art which is focused on ameliorating or mitigating the systems of nerve damage, such as pain. Moreover, in contrast to much of the conventional art that relies on activation of PKC-β, the methods described herein are based on activation of PKC-α and PKC-ε.

More particularly, the method includes administering (e.g., intravenously or orally) to the subject a pharmaceutically effective amount of a bryostatin compound or analogue thereof (e.g., a bryolog) or a polyunsaturated fatty acid (PUFA) or derivative thereof on a weekly dosage regimen to result in at least partial repair or regeneration of damaged nerves in the subject, wherein the weekly dosage regimen comprises a dosage of about 10 to about 60 μg/m² once a week for at least two, three, or four weeks, wherein the at least two, three, or four weeks of dosing are done consecutively or intermittently. In some embodiments, the weekly dosage regimen comprises a dosage of about 10 to about 60 μg/m² once a week for at least two consecutive weeks followed by a dosage of about 10 to about 60 μg/m² once a week for at least two weeks done intermittently. In some embodiments, the dosage is, in each instance, independently about 10 to about 50 mg/m² or 40 mg/m².

In other embodiments, the weekly dosage regimen is done intermittently as follows: (i) a first period in which the subject is administered the bryostatin compound or analogue thereof or a polyunsaturated fatty acid (PUFA) or derivative thereof in a dosage of about 10 to about 60 μg/m² once a week for 1-3 or 2-3 weeks consecutively, (ii) a second period of 1-3 weeks in which the subject is not administered a bryostatin compound or analogue thereof or a polyunsaturated fatty acid (PUFA) or derivative thereof, and (iii) a third period in which the subject is administered the bryostatin compound or analogue thereof or a polyunsaturated fatty acid (PUFA) or derivative thereof in a dosage of about 10 to about 60 μg/m² once a week for at least one week, two weeks, or three weeks done consecutively or intermittently.

In other embodiments, the weekly dosage regimen is done intermittently as follows: (i) a first period in which the subject is administered the bryostatin compound or analogue thereof or a polyunsaturated fatty acid (PUFA) or derivative thereof in a loading dosage of 13-20 μg/m² (or about 15 μg/m²) once a week for 1-3 weeks consecutively, (ii) a second period of 1-3 weeks in which the subject is not administered a bryostatin compound or analogue thereof or a polyunsaturated fatty acid (PUFA) or derivative thereof, and (iii) a third period in which the subject is administered the bryostatin compound or analogue thereof or a polyunsaturated fatty acid (PUFA) or derivative thereof in a dosage of about 10-17 μg/m² (or about 12 μg/m²) once a week for at least two weeks done consecutively or intermittently, wherein the loading dosage is at least 3 μg/m² higher than the dosage in the third period.

In other embodiments, the weekly dosage regimen is done intermittently as follows: (i) a first period in which the subject is administered the bryostatin compound or analogue thereof or a polyunsaturated fatty acid (PUFA) or derivative thereof in a loading dosage of 20-30 μg/m² (or about 24 μg/m²) once a week for 1-3 weeks consecutively, (ii) a second period of 1-3 weeks in which the subject is not administered a bryostatin compound or analogue thereof or a polyunsaturated fatty acid (PUFA) or derivative thereof, and (iii) a third period in which the subject is administered the bryostatin compound or analogue thereof or a polyunsaturated fatty acid (PUFA) or derivative thereof in a dosage of about 15-27 μg/m² (or about 20 μg/m²) once a week for at least two weeks done consecutively or intermittently, wherein the loading dosage is at least 3 μg/m² higher than the dosage in the third period.

In other embodiments, the weekly dosage regimen is done intermittently as follows: (i) a first period in which the subject is administered the bryostatin compound or analogue thereof or a polyunsaturated fatty acid (PUFA) or derivative thereof in a loading dosage of 30-60 μg/m² (48 μg/m²) once a week for 1-3 weeks consecutively, (ii) a second period of 1-3 weeks in which the subject is not administered a bryostatin compound or analogue thereof or a polyunsaturated fatty acid (PUFA) or derivative thereof, and (iii) a third period in which the subject is administered the bryostatin compound or analogue thereof or a polyunsaturated fatty acid (PUFA) or derivative thereof in a dosage of about 27-57 μg/m² (40 μg/m²) once a week for at least two weeks done consecutively or intermittently, wherein the loading dosage is at least 3 μg/m² higher than the dosage in the third period.

### DETAILED DESCRIPTION

The present disclosure is directed to methods for treating nerve damage in a living subject. The subject is typically human, but may be, e.g., a horse, dog, or monkey. The nerve damage may have any etiology and may be associated with any condition. The subject may have, for example, spinal cord injury, cochlear nerve damage, or nerve damage to direct peripheral nerves, such as in the extremities (e.g., carpal tunnel syndrome). The nerve damage may be a result of, for example, an injury (e.g., physical injury or exposure to a neurotoxin), autoimmune disorder, or adverse effect from another disease or condition (e.g., diabetes or amyotrophic lateral sclerosis, i.e., ALS). Moreover, the nerve damage may be deemed acute, sub-acute, or nascent. In some embodiments, the nerve damage is a result of a neurodegenerative disorder characterized by sudden or progressive degeneration of upper and/or lower motor neurons in the cortex, brainstem, and/or spinal cord, such as ALS. The subject may have any of the diseases or conditions mentioned above at the time of treatment for nerve damage or to prevent nerve damage. In particular embodiments, a therapeutic agent or compound, such as a bryostatin or analogue thereof (e.g., a bryolog compound) or a polyunsaturated fatty acid (PUFA) or derivative thereof is administered in an efficacious amount to stop, mitigate, repair, regenerate, reverse, or prevent nerve damage in the subject.

As used herein, “protein kinase C activator” or “PKC activator” refers to a substance that increases the rate of the reaction catalyzed by PKC. PKC activators can be non-specific or specific activators. A specific activator activates one PKC isoform, e.g., PKC-ε (epsilon), to a greater detectable extent than another PKC isoform. Protein kinase C (PKC) is one of the largest gene families of protein kinase. Several PKC isozymes are expressed in the brain, including PKCα, PKCβ1, PKCβII, PKCδ, PKCε, and PKCγ. PKC is primarily a cytosolic protein, but with stimulation it translocates to the membrane.

PKC activators have been associated with various diseases and conditions. For example, PKC has been shown to be involved in numerous biochemical processes relevant to AD, and PKC activators have demonstrated neuroprotective activity in animal models of AD. PKC activation has a crucial role in learning and memory enhancement, and PKC activators have been shown to increase memory and learning (Sun and Alkon, Eur J Pharmacol. 2005; 512:43-51; Alkon et al., Proc Natl Acad Sci USA. 2005; 102:16432-16437). PKC activation also has been shown to induce synaptogenesis in rat hippocampus, suggesting the potential of PKC-mediated antiapoptosis and synaptogenesis during conditions of neurodegeneration (Sun and Alkon, Proc Natl Acad Sci USA. 2008; 105(36): 13620-13625). In fact, synaptic loss appears to be a pathological finding in the brain that is closely correlated with the degree of dementia in AD patients. PKC activation has further been shown to protect against traumatic brain injury-induced learning and memory deficits (Zohar et al., Neurobiology of Disease, 2011, 41: 329-337), has demonstrated neuroprotective activity in animal models of stroke (Sun et al., Eur. J. Pharmacol., 2005, 512: 43-51), and has shown neuroprotective activity in animal models of depression (Sun et al., Eur. J. Pharmacol., 2005, 512: 43-51).

Neurotrophins, particularly brain-derived neurotrophic factor (BDNF) and nerve growth factor (NGF), are key growth factors that initiate repair and regrowth of damaged neurons and synapses. Activation of some PKC isoforms, particularly PKCε and PKCα, protect against neurological injury, most likely by upregulating the production of neurotrophins such as BDNF (Weinreb et al., FASEB Journal. 2004; 18:1471-1473). The activation of PKCε also increases brain postsynaptic density anchoring protein (PSD-95) which is an important marker for synaptogenesis.

As used herein, the word “synaptogenesis” refers to a process involving the formation of synapses. As used herein, the word “synaptic networks” refer to a multiplicity of neurons and synaptic connections between the individual neurons. Synaptic networks may include extensive branching with multiple interactions. Synaptic networks can be recognized, for example, by confocal visualization, electron microscopic visualization, and electrophysiologic recordings. Notably, the PKC activating therapeutic compounds described herein are capable of promoting synaptogenesis when administered in efficacious amounts, and the synaptogenesis can be instrumental in the repair and regeneration of nerve tissue.

The phrase “pharmaceutically acceptable” refers to molecular entities and compositions that are physiologically tolerable and do not typically produce adverse reactions when administered to a subject. The pharmaceutically acceptable substance is typically approved by a regulatory agency or listed in the U.S. Pharmacopeia or other generally recognized pharmacopeia for use in animals, and more particularly in humans. The term “pharmaceutically acceptable carrier” generally refers to a liquid or solid chemical substance in which the active ingredient may be combined and which, following the combination, can be used to deliver the active ingredient to a subject. The carrier can also be, for example, a diluent, adjuvant, excipient, or vehicle for the compound being administered.

The term “therapeutically effective amount” refers to an amount of a therapeutic agent, which is a PKC activator, that results in a measurable or observable therapeutic response. A therapeutic response may be, for example, any response that a person of sound medical adjustment (e.g., a clinician or physician) will recognize as an effective response to the therapy, including improvement of symptoms and surrogate clinical markers. Thus, a therapeutic response will generally be a mitigation, amelioration, or inhibition of one or more symptoms of nerve damage. A measurable therapeutic response may also include a finding that the nerve damage has been prevented or has a delayed onset or is otherwise attenuated by the therapeutic agent.

The term “subject,” as used herein, refers to a human or other mammal in need of treatment with the therapeutic agent (bryostatin or analogue thereof or PUFA or derivative thereof). The subject may be, for example, a human having nerve damage. Some examples of mammals other than humans that may be treated include dogs, cats, monkeys, and apes. In different embodiments, the subject may have an age of at least 20, 30, 40, 50, 60, 70, or 80 years.

The terms “approximately” and “about” mean to be nearly the same as a referenced number or value including an acceptable degree of error for the quantity measured given the nature or precision of the measurements. As used herein, the terms “approximately” and “about” are generally understood to encompass ±20% or ±10% of a specified amount, frequency or value. Numerical quantities given herein are approximate unless stated otherwise, meaning that the term “about” or “approximately” can be inferred when not expressly stated. For example, the term “about 20 μg” may be interpreted as precisely that amount or as being within a margin of 16-24 μg or 18-22 μg.

The terms “administer,” “administration,” or “administering,” as used herein, refer to (1) providing, giving, dosing and/or prescribing by either a health practitioner or his/her authorized agent or under his/her direction a composition according to the disclosure, and (2) putting into, taking, or consuming by the patient or person himself or herself, a composition according to the disclosure. As used herein, “administration” of a composition includes any route of administration, including oral, intravenous, subcutaneous, intraperitoneal, and intramuscular.

The phrase “weekly dosing regimen” is used when the subject is administered a single dose of a therapeutic agent (drug) each week for a specified number of weeks, wherein the number of weeks may be consecutive or intermittent. The term “intermittent” (or “spaced dosing”) indicates that the weekly dosing regimen includes at least one week in which no bryostatin compound or analogue thereof or PUFA or derivative thereof is administered, with one or more weekly administrations occurring before and after the gap in administration. For example, the subject may receive a single dose of a therapeutic agent each week for three consecutive weeks. An example of an intermittent three-week administration includes the following: administering to the subject a single dose of the therapeutic agent for one week, followed by a week of no administration, followed by two weeks in which the subject is administered the therapeutic agent once per week. In some embodiments, intermittent dosing of the therapeutic agent results in restoring or upregulating BDNF, increasing the postsynaptic density of the anchoring protein PSD-95, and/or lowering or preventing the downregulation of PCK-ε.

The spaced or intermittent dosing regimen may entail, for example, administering the therapeutic agent to the subject once a week for two or three consecutive weeks, followed by cessation of administration or dosing for two or three consecutive weeks. In further embodiments, the administration may continue in alternating intervals of administering the therapeutic agent once a week for two or three consecutive weeks, followed by cessation of administration or dosing for two or three consecutive weeks, and continuing those alternating intervals over a period of about 4 months, about 8 months, about 1 year, about 2 years, about 5 years, or otherwise for the duration of therapy with the therapeutic agent.

The therapeutic agent (e.g., a bryostatin, PUFA, or analogue or derivative thereof) may be administered according to any suitable dosing schedule or regimen. In some embodiments, the therapeutic agent may be administered in an amount ranging from about 0.01 μg/m² to about 100 μg/m², which can be by, e.g., i.v. or oral administration. In different embodiments, the amount administered is precisely, about, up to, or less than 0.01 μg/m², 0.05 μg/m², 0.1 μg/m², 0.5 μg/m², 1 μg/m², 5 μg/m², 10 μg/m², 15 μg/m², 20 μg/m², 25 μg/m², 30 μg/m², 35 μg/m², 40 μg/m², 45 μg/m², 50 μg/m², 55 μg/m², 60 μg/m², 65 μg/m², 70 μg/m², 75 μg/m², 80 μg/m², 85 μg/m², 90 μg/m², 95 μg/m², or 100 μg/m², or an amount within a range bounded by any two of the foregoing amounts, e.g., 0.01-100 μg/m², 0.1-100 μg/m², 1-100 μg/m², 5-100 μg/m², 10-100 μg/m², 20-100 μg/m², 0.01-50 μg/m², 0.1-50 μg/m², 1-50 μg/m², 5-50 μg/m², 10-50 μg/m², 20-50 μg/m², 0.01-40 μg/m², 0.1-40 μg/m², 1-40 μg/m², 5-40 μg/m², 10-40 μg/m², 20-40 μg/m², 0.01-30 μg/m², 0.1-30 μg/m², 1-30 μg/m², 5-30 μg/m², 10-30 μg/m², 20-30 μg/m², 0.01-20 μg/m², 0.1-20 μg/m², 1-20 μg/m², 5-20 μg/m², or 10-20 μg/m². In particular embodiments, the amount may range from about 10-40 μg/m², or more particularly, about 15 μg/m², about 20 μg/m², about 25 μg/m², about 30 μg/m², about 35 μg/m², or about 40 μg/m², or about 45 μg/m², or about 50 μg/m², or an amount within a range bounded by any two of the foregoing values. Notably, any of the amounts above or below expressed as “μg/m²” may alternatively be interpreted in terms of micrograms (μg) or micrograms per 50 kg body weight “μg/50 kg”. For example, 25 μg/m² may be interpreted as 25 μg or 25 μg/50 kg. In particular embodiments, any of the foregoing amounts (which may be within any of the exemplary ranges disclosed above) may be administered per day, every other day, once every three days, precisely or at least once per week, precisely or at least once every two weeks, and any of the foregoing administrations may be continued as a dosing regimen for at least one, two, three, four, five, six, seven, eight, nine, ten, eleven, or twelve weeks, or for at least four, five, six, seven, eight, nine, ten, eleven, or twelve months.

In some embodiments, the therapeutic agent is administered as a dose in the range of about 0.01 to 100 μg/m²/week, which can be by, e.g., i.v. or oral administration. For example, the dose may be administered each week in a range of about 0.01 to about 25 μg/m²/week; about 1 to about 20 μg/m²/week, about 5 to about 20 μg/m²/week, or about 10 to about 20 μg/m²/week. In particular embodiments, the dose may be about or less than, for example, 5 μg/m²/week, 10 μg/m²/week, 15 μg/m²/week, 20 μg/m²/week, 25 μg/m²/week, or 20 μg/m²/week. Any of the foregoing dosages may be administered over a suitable time period, e.g., three weeks, four weeks, (approximately 1 month), two months, three months (approximately 12 or 13 weeks), four months, five months, six months, or a year. Notably, any of the amounts above or below expressed as “μg/m²” may alternatively correspond to micrograms (μg) or micrograms per 50 kg body weight “μg/50 kg” per dosage or amount per week.

In some embodiments, the therapeutic agent (e.g., a bryostatin, bryolog, PUFA, or analogue or derivative thereof) may be administered in an amount of 8-12 times (8-12×) or about 10× any of the above disclosed amounts or ranges thereof, which can be by, e.g., i.v. or oral administration. For example, in some embodiments, the therapeutic agent may be administered in an amount ranging from about 0.1-1500 μg/m²/week, or alternatively about 0.1-1000 μg/m²/week. In different embodiments, the amount administered is precisely or about, for example, 0.1, 0.2, 0.5, 1, 2, 5, 10, 20, 50, 100, 200, 500, 800, 1000, 1200, or 1500 μg/m²/week, or an amount within a range bounded by any two of the foregoing values, e.g., 0.1-800 μg/m²/week, 0.1-500 μg/m²/week, 0.1-200 μg/m²/week, 0.1-100 μg/m²/week, 0.5-1500 μg/m²/week, 0.5-1200 μg/m²/week, 0.5-1000 μg/m²/week, 0.5-800 μg/m²/week, 0.5-500 μg/m²/week, 0.5-200 μg/m²/week, 0.5-100 μg/m²/week, 1-1500 μg/m²/week, 1-1200 μg/m²/week, 1-1000 μg/m²/week, 1-500 μg/m²/week, 10-1500 μg/m²/week, 10-1200 μg/m²/week, 10-1000 μg/m²/week, 50-1500 μg/m²/week, 50-1200 μg/m²/week, 50-1000 μg/m²/week, 100-1500 μg/m²/week, 100-1200 μg/m²/week, 100-1000 μg/m²/week, 100-800 μg/m²/week, 200-1500 μg/m²/week, 200-1200 μg/m²/week, 200-1000 μg/m²/week, 200-800 μg/m²/week, 500-1500 μg/m²/week, 500-1200 μg/m²/week, 500-1000 μg/m²/week, 500-800 μg/m²/week, 800-1500 μg/m²/week, 800-1200 μg/m²/week, 800-1000 μg/m²/week, or 1000-1500 μg/m²/week, any of which may be by i.v., or oral administration.

In some embodiments, any of the above specific values or ranges thereof is instead administered twice per week or three times per week. A dosing regimen of 0.1-1000 μg/m² given twice a week corresponds to 0.2-2000 μg/m²/week. Similarly, a dosing regimen of 0.1-1500 μg/m² twice a week corresponds to 0.2-3000 μg/m²/week. Thus, in some embodiments, the therapeutic agent may be administered twice a week to result in a total amount of 1-3000 μg/m²/week, 1-2500 μg/m²/week, 1-2000 μg/m²/week, 10-3000 μg/m²/week, 10-2500 μg/m²/week, 10-2000 μg/m²/week, 50-3000 μg/m²/week, 50-2500 μg/m²/week, 50-2000 μg/m²/week, 100-3000 μg/m²/week, 100-2500 μg/m²/week, 100-2000 μg/m²/week, 500-3000 μg/m²/week, 500-2500 μg/m²/week, 500-2000 μg/m²/week, 1000-3000 μg/m²/week, 1000-2500 μg/m²/week, or 1000-2000 μg/m²/week, any of which may be by i.v., or oral administration. When given twice a week, the administration is typically given in two equal portions (e.g., two 500, 1000, or 1500 μg/m² administrations) spaced one, two, three, four, or five days apart.

In some embodiments, any of the amounts disclosed in this application are alternatively administered once daily, every two days, every three days, every four days, or every five days instead of once a week. For example, the therapeutic agent may be administered in an amount of 0.1, 0.2, 0.5, 1, 2, 5, 10, 20, 50, 100, 200, 500, 800, 1000, 1200, 1500, 2000, or 2500 μg/m² or any range therein, which may be selected from any of the ranges disclosed in this application, daily, twice daily, every two days, every three days, every four days, or every five days, which may be perpetuated over a period of precisely or at least, for example, two weeks, three weeks, four weeks, six weeks, eight weeks, ten weeks, twelve weeks, fourteen weeks, sixteen weeks, eighteen weeks, twenty weeks, 3 months, 4 months, 5 months, 6 months, or a year. In some embodiments, the therapeutic agent (e.g., a bryostatin, PUFA, or analogue or derivative thereof) is administered in an amount of precisely or about 20 μg, 30 μg, or 40 μg (20 μg/m², 30 μg/m², or 40 μg/m²) every week or every two weeks for a total period of time of, e.g., four weeks, (approximately 1 month), five weeks, six weeks, eight weeks, ten weeks, twelve weeks, four months, five months, six months, or a year. The administration may alternatively start with an initial (loading) single higher amount (e.g., 10%, 15%, 20%, or 25% higher amount than successive administrations). For example, in some embodiments, the therapeutic agent may be administered in an amount of precisely or about 15 μg, 24 μg, or 48 μg for the first week or first two or three consecutive weeks followed by administrations of 12 μg, 20 μg or 40 μg, respectively, every week or alternately every two or three weeks for at least four weeks (approximately 1 month), six weeks, eight weeks, ten weeks, twelve weeks, fifteen weeks, eighteen weeks, or for at least three months, four months, five months, six months, or a year. The term “alternately,” as used herein, indicates a period of time in which the therapeutic agent is not being administered. For example, “alternately every two or three weeks” indicates, respectively, regular one-week periods of no administration or regular two-week periods of no administration (also referred to herein as “1 on/1 off” and “1 on/2 off” dosing regimens). Other alternating dosing regimens are possible, including, for example, “2 on/1 off”, “2 on/2 off”, “1 on/3 off”, “2 on/3 off”, “3 on/3 off”, “3 on/1 off”, and “3 on/2 off”. Notably, any of the amounts above or below expressed as μg may alternatively be interpreted in terms of μg/m² or micrograms per 50 kg body weight “μg/50 kg”.

In some embodiments, the weekly dosage regimen comprises a dosage of the therapeutic agent of precisely or about 10, 20, 30, 40, 50, or 60 μg/m² once a week for at least two, three, four, five, or six weeks, wherein the at least two, three, four, five, or six weeks of dosing are done consecutively or intermittently. In some embodiments, the weekly dosage regimen comprises a dosage in a range bounded by any of the values provided above, e.g., about 10 to about 60 μg/m², or about 10 to about 50 μg/m², or about 10 to about 40 μg/m², or about 10 to about 30 μg/m², or about 10 to about 20 μg/m², or about 20 to about 60 μg/m², or about 20 to about 50 μg/m², or about 20 to about 40 μg/m², or about 30 to about 60 μg/m², or about 30 to about 50 μg/m², or about 40 to about 60 μg/m², once a week for at least two, three, four, five, or six weeks, wherein the at least two, three, four, five, or six weeks of dosing are done consecutively or intermittently as described above.

In some embodiments, the weekly dosage regimen is done intermittently as follows: (i) a first period in which the subject is administered the bryostatin compound or analogue thereof or PUFA or derivative thereof in a dosage of about 10, 20, 30, 40, 50, or 60 μg/m² (or any range therein, as provided above) once a week for 1-3 weeks consecutively, (ii) a second period of 1, 2, or 3 weeks (or 1-2, 1-3, or 2-3 weeks) in which the subject is not administered a bryostatin compound or analogue thereof or PUFA or derivative thereof, and (iii) a third period in which the subject is administered the bryostatin compound or analogue thereof or PUFA or derivative thereof in a dosage of about 10, 20, 30, 40, 50, or 60 μg/m² (or any range therein, as provided above) once a week for precisely or at least one week, two weeks, three weeks, four weeks, five weeks, or six weeks, done consecutively or intermittently. In particular embodiments, the dosage administered in the first and/or third week is independently within a range of about 10 to about 60 μg/m², or about 10 to about 50 μg/m², or about 10 to about 40 μg/m², or about 10 to about 30 μg/m², or about 10 to about 20 μg/m², or about 20 to about 60 μg/m², or about 20 to about 50 μg/m², or about 20 to about 40 μg/m², or about 30 to about 60 μg/m², or about 30 to about 50 μg/m², or about 40 to about 60 μg/m². In some embodiments, in the third period, the bryostatin compound or analogue thereof or PUFA or derivative thereof is administered on an intermittent schedule by administering once every two weeks or once every three weeks, wherein only the weeks in which a dosage is administered are counted in the number of weeks. For example, the term “administering once a week for four weeks intermittently” may correspond to administering once every two weeks for a total of eight weeks or administering once every three weeks for a total of twelve weeks.

In a first set of particular embodiments, the weekly dosage regimen is done intermittently as follows: (i) a first period in which the subject is administered the bryostatin compound or analogue thereof or PUFA or derivative thereof in a loading dosage of 13-20 μg/m² (or more particularly, precisely or about 13, 15, 17, or 20 μg/m²) once a week for 1, 2, or 3 weeks (or 1-2, 1-3, or 2-3 weeks) consecutively, (ii) a second period of 1, 2, or 3 weeks (or 1-2, 1-3, or 2-3 weeks) in which the subject is not administered a bryostatin compound or analogue thereof or PUFA or derivative thereof, and (iii) a third period in which the subject is administered the bryostatin compound or analogue thereof or PUFA or derivative thereof in a dosage of about 10-17 μg/m² (or more particularly, precisely or about 10, 12, 15, or 17 μg/m²) once a week for precisely or at least two, three, four, five, or six weeks done consecutively or intermittently, wherein the loading dosage is at least 3, 4, 5, or 6 μg/m² higher than the dosage in the third period. In some embodiments, in the third period, the bryostatin compound or analogue thereof or PUFA or derivative thereof is administered on an intermittent schedule by administering once every two weeks or once every three weeks, as described above.

In a second set of particular embodiments, the weekly dosage regimen is done intermittently as follows: i) a first period in which the subject is administered the bryostatin compound or analogue thereof or PUFA or derivative thereof in a loading dosage of 20-30 μg/m² (or more particularly, precisely or about 20, 22, 24, 27, or 30 μg/m²) once a week for 1, 2, or 3 weeks (or 1-2, 1-3, or 2-3 weeks) consecutively, (ii) a second period of 1, 2, or 3 weeks (or 1-2, 1-3, or 2-3 weeks) in which the subject is not administered a bryostatin compound or analogue thereof or PUFA or derivative thereof, and (iii) a third period in which the subject is administered the bryostatin compound or analogue thereof or PUFA or derivative thereof in a dosage of about 15-27 μg/m² (or more particularly, precisely or about 15, 17, 20, 24, or 27 μg/m²) once a week for precisely or at least two, three, four, five, or six weeks done consecutively or intermittently, wherein the loading dosage is at least 3, 4, 5, or 6 μg/m² higher than the dosage in the third period. In some embodiments, in the third period, the bryostatin compound or analogue thereof or PUFA or derivative thereof is administered on an intermittent schedule by administering once every two weeks or once every three weeks, as described earlier above.

In a third set of particular embodiments, the weekly dosage regimen is done intermittently as follows: (i) a first period in which the subject is administered the bryostatin compound or analogue thereof or PUFA or derivative thereof in a loading dosage of 30-60 μg/m² (or more particularly, precisely or about 30, 32, 35, 38, 40, 42, 45, 48, 50, 52, 55, 58, or 60 μg/m²) once a week for 1, 2, or 3 weeks (or 1-2, 1-3, or 2-3 weeks) consecutively, (ii) a second period of 1, 2, or 3 weeks (or 1-2, 1-3, or 2-3 weeks) in which the subject is not administered a bryostatin compound or analogue thereof or PUFA or derivative thereof, and (iii) a third period in which the subject is administered the bryostatin compound or analogue thereof or PUFA or derivative thereof in a dosage of about 27-57 μg/m² (or more particularly, precisely or about 27, 30, 32, 35, 37, 40, 42, 45, 47, 50, 52, 55, or 57 μg/m²) once a week for at least two weeks done consecutively or intermittently, wherein the loading dosage is at least 3, 4, 5, or 6 μg/m² higher than the dosage in the third period. In some embodiments, in the third period, the bryostatin compound or analogue thereof or PUFA or derivative thereof is administered on an intermittent schedule by administering once every two weeks or once every three weeks, as described above.

For purposes of the present invention, the administration of the therapeutic agent should result in at least a stabilization, and more preferably, a restoration or regeneration of nerve tissue. The administration may alternatively prevent such nerve damage from occurring or from worsening. In some embodiments, administration of the therapeutic agent results in an increase in the number of fully mature mushroom spine synapses. In other embodiments, administration of the therapeutic agent results in at least partial or full restoration of mature mushroom spines or mushroom spine synapses.

In some embodiments, before administering the therapeutic agent, the subject is first confirmed to have nerve damage. For example, the subject may be confirmed to have spinal cord injury, cochlear nerve damage, or nerve damage to direct peripheral nerves. The testing and confirmation of such conditions can be done by means well known in the medical arts.

In some embodiments, the therapeutic agent is or includes a bryostatin. Some examples of bryostatins include bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, or bryostatin-18.

In a particular embodiment, the bryostatin is Bryostatin-1 (shown below).

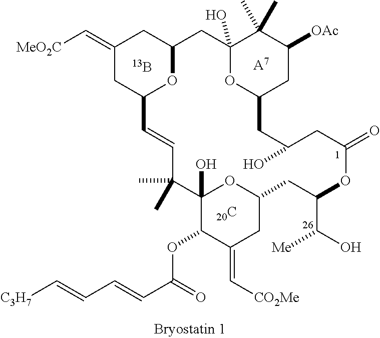

In another particular embodiment, the bryostatin is Bryostatin-2 (shown below; R═COC₇H₁₁, R′═H).

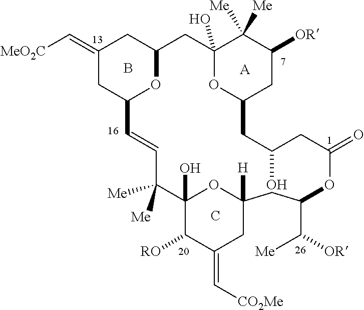

In other embodiments, the therapeutic agent is a bryostatin analogue, such as neristatin (e.g., neristatin-1) or a bryolog. Bryostatin analogs are described, for example, in U.S. Pat. Nos. 6,624,189 and 7,256,286. Bryologs can be chemically synthesized or produced by certain bacteria. Different bryologs exist that modify, for example, the rings A, B, and C (see Bryostatin-1, figure shown above) as well as the various substituents. Generally, bryologs are considered less specific and less potent than bryostatin but are easier to prepare.

Table 1 summarizes structural characteristics of several bryologs and their affinity for PKC (ranging from 0.25 nM to 10 μM). While Bryostatin-1 has two pyran rings and one 6-membered cyclic acetal, in most bryologs one of the pyrans of Bryostatin-1 is replaced with a second 6-membered acetal ring. This modification may reduce the stability of bryologs, relative to Bryostatin-1, for example, in strong acid or base, but has little significance at physiological pH. Bryologs also tend to have a lower molecular weight (ranging from about 600 g/mol to 755 g/mol), as compared to Bryostatin-1 (988), a property which may facilitate transport across the blood-brain barrier.

TABLE 1
Bryologs

| | PKC Affinity | | |
|---|---|---|---|
| Name | (nM) | MW | Description |
| Bryostatin-1 | 1.35 | 988 | 2 pyran + 1 cyclic acetal + macrocycle |
| Analog 1 | 0.25 | 737 | 1 pyran + 2 cyclic acetal + macrocycle |
| Analog 2 | 6.50 | 723 | 1 pyran + 2 cyclic acetal + macrocycle |
| Analog 7a | — | 642 | 1 pyran + 2 cyclic acetals + macrocycle |
| Analog 7b | 297 | 711 | 1 pyran + 2 cyclic acetals + macrocycle |
| Analog 7c | 3.4 | 726 | 1 pyran + 2 cyclic acetals + macrocycle |
| Analog 7d | 10000 | 745 | 1 pyran + 2 cyclic acetals + macrocycle, |
| | | | acetylated |
| Analog 8 | 8.3 | 754 | 2 cyclic acetals + macrocycle |
| Analog 9 | 10000 | 599 | 2 cyclic acetals |

Analog 1 exhibits the highest affinity for PKC. Wender et al., Curr. Drug Discov. Technol. (2004), vol. 1, pp. 1-11; Wender et al. Proc. Natl. Acad. Sci. (1998), vol. 95, pp. 6624-6629; Wender et al., J. Am. Chem. Soc. (2002), vol. 124, pp. 13648-13649, each incorporated by reference herein in their entireties. Only Analog 1 exhibits a higher affinity for PKC than Bryostatin-1. Analog 2, which lacks the A ring of Bryostatin-1, is the simplest analog that maintains high affinity for PKC. In addition to the active bryologs, Analog 7d, which is acetylated at position 26, has virtually no affinity for PKC.

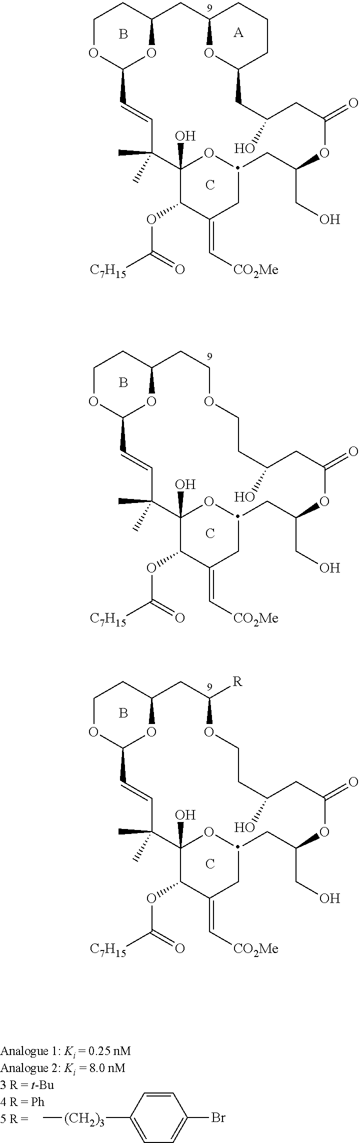

B-ring bryologs may also be used in the present disclosure. These synthetic bryologs have affinities in the low nanomolar range. Wender et al., Org Lett. (2006), vol. 8, pp. 5299-5302, incorporated by reference herein in its entirety. B-ring bryologs have the advantage of being completely synthetic, and do not require purification from a natural source.

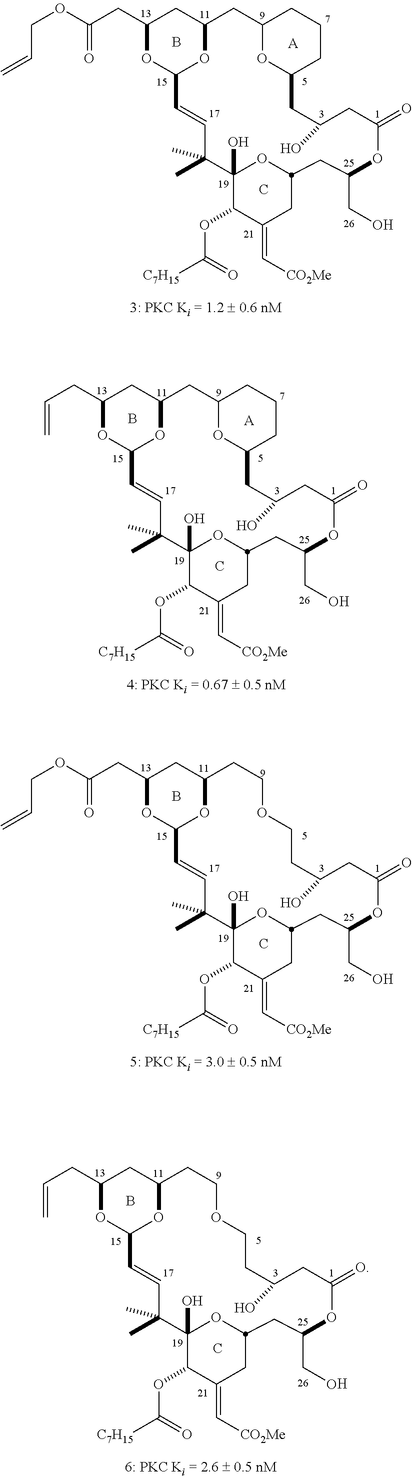

A third class of suitable bryostatin analogs are the A-ring bryologs. These bryologs generally have slightly lower affinity for PKC than Bryostatin-1 (6.5 nM, 2.3 nM, and 1.9 nM for bryologs 3, 4, and 5, respectively) and a lower molecular weight. A-ring substituents are important for non-tumorigenesis.

As used herein, the term “fatty acid” refers to a compound composed of a hydrocarbon chain and ending in a free acid, an acid salt, or an ester. When not specified, the term “fatty acid” is meant to encompass all three forms. Those skilled in the art understand that certain expressions are interchangeable. For example, “methyl ester of linolenic acid” is the same as “linolenic acid methyl ester,” which is the same as “linolenic acid in the methyl ester form.”

As used herein, the term “cyclopropanated” or “CP” refers to a compound wherein at least one carbon-carbon double bond in the molecule has been replaced with a cyclopropane ring. The cyclopropyl group may be in cis or trans configuration. Unless otherwise indicated, the cyclopropyl group is in the cis configuration. Compounds with multiple carbon-carbon double bonds have many cyclopropanated forms. For example, a polyunsaturated compound in which only one double bond has been cyclopropanated is herein referred to as being in “CP1 form.” Similarly, “CP6 form” indicates that six double bonds are cyclopropanated. Docosahexaenoic acid (“DHA”) methyl ester has six carbon-carbon double bonds, and thus, can have 1-6 cyclopropane rings.

Shown below are the CP1 and CP6 forms. With respect to compounds that are not completely cyclopropanated (e.g. IDA-CP1), the cyclopropane group(s) can occur at any of the carbon-carbon double bonds.

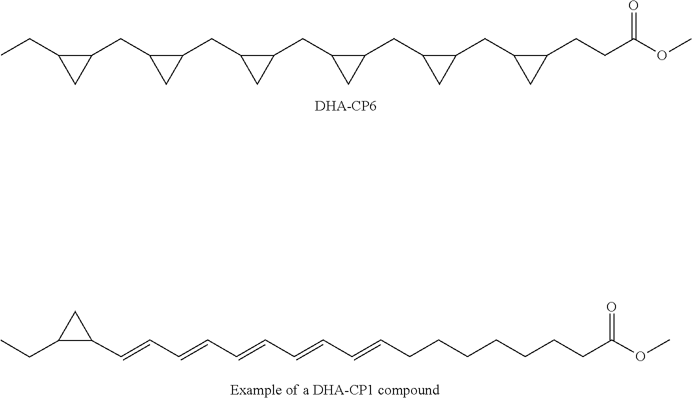

In some embodiments, the fatty acid is a polyunsaturated fatty acid (“PUFA”). These compounds are essential components of the nervous system and have numerous health benefits. In general, PUFAs increase membrane fluidity, rapidly oxidize to highly bioactive products, produce a variety of inflammatory and hormonal effects, and are rapidly degraded and metabolized. The inflammatory effects and rapid metabolism is likely the result of their active carbon-carbon double bonds.

In one embodiment, the PUFA is selected from linoleic acid (shown below).

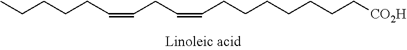 [SMILES: CCCCC/C=C\C/C=C\CCCCCCCC(=O)O]

The PKC activator may also be a PUFA or MUFA derivative. In particular embodiments, the PUFA or MUFA derivative is a cyclopropanated PUFA or MUFA. Certain cyclopropanated PUFAs, such as DCPLA (i.e., linoleic acid with cyclopropane at both double bonds), may be able to selectively activate PKC-ε. See Journal of Biological Chemistry, 2009, 284(50): 34514-34521; see also U.S. Patent Application Publication No. 2010/0022645 A1. Like their parent molecules, PUFA derivatives are thought to activate PKC by binding to the PS site.

Cyclopropanated fatty acids exhibit low toxicity and are readily imported into the brain where they exhibit a long half-life (t_{1/2}). Conversion of the double bonds into cyclopropane rings prevents oxidation and metabolism to inflammatory byproducts and creates a more rigid U-shaped 3D structure that may result in greater PKC activation. Moreover, this U-shape may result in greater isoform specificity. For example, cyclopropanated fatty acids may exhibit potent and selective activation of PKC-ε.

The Simmons-Smith cyclopropanation reaction is an efficient way of converting double bonds to cyclopropane groups. This reaction, acting through a carbenoid intermediate, preserves the cis-stereochemistry of the parent molecule. Thus, the PKC-activating properties are increased while metabolism into other molecules, such as bioreactive eicosanoids, thromboxanes, or prostaglandins, is prevented.

A particular class of PKC-activating fatty acids is omega-3 PUFA derivatives. In at least one embodiment, the omega-3 PUFA derivatives are selected from cyclopropanated docosahexaenoic acid, cyclopropanated eicosapentaenoic acid, cyclopropanated rumelenic acid, cyclopropanated parinaric acid, and cyclopropanated linolenic acid (CP3 form shown below).

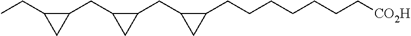 [SMILES: CCC1CC1CC1CC1CC1CC1CCCCCCCC(=O)O]

Another class of PKC-activating fatty acids is omega-6 PUFA derivatives. In at least one embodiment, the omega-6 PUFA derivatives are selected from cyclopropanated linoleic acid (“DCPLA,” CP2 form shown below),

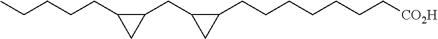 [SMILES: CCCCCC1CC1CC1CC1CCCCCCCC(=O)O]

cyclopropanated arachidonic acid, cyclopropanated eicosadienoic acid, cyclopropanated dihomo-gamma-linolenic acid, cyclopropanated docosadienoic acid, cyclopropanated adrenic acid, cyclopropanated calendic acid, cyclopropanated docosapentaenoic acid, cyclopropanated jacaric acid, cyclopropanated pinolenic acid, cyclopropanated podocarpic acid, cyclopropanated tetracosatetraenoic acid, and cyclopropanated tetracosapentaenoic acid.

Vernolic acid is a naturally occurring compound. However, it is an epoxyl derivative of linoleic acid and therefore, as used herein, is considered an omega-6 PUFA derivative. In addition to vernolic acid, cyclopropanated vernolic acid (shown below) is an omega-6 PUFA derivative.

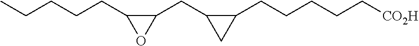 [SMILES: CCCCCC1OC1CC1CC1CCCCCC(=O)O]

Another class of PKC-activating fatty acids is the class of omega-9 PUFA derivatives. In at least one embodiment, the omega-9 PUFA derivatives are selected from cyclopropanated eicosenoic acid, cyclopropanated mead acid, cyclopropanated erucic acid, and cyclopropanated nervonic acid.

Yet another class of PKC-activating fatty acids is the class of monounsaturated fatty acid (“MUFA”) derivatives. In at least one embodiment, the MUFA derivatives are selected from cyclopropanated oleic acid (shown below),

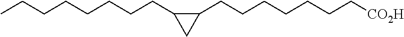 [SMILES: CCCCCCCCC1CC1CCCCCCCC(=O)O]

and cyclopropanated elaidic acid (shown below).

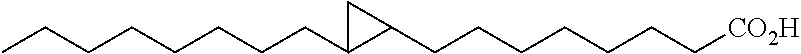 [SMILES: CCCCCCCCC1CC1CCCCCCCC(=O)O]

PKC-activating MUFA derivatives include epoxylated compounds such as trans-9,10-epoxystearic acid (shown below).

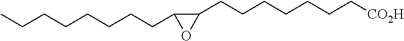 [SMILES: CCCCCCCCC1OC1CCCCCCCC(=O)O]

Another class of PKC-activating fatty acids is the class of omega-5 and omega-7 PUFA derivatives. In at least one embodiment, the omega-5 and omega-7 PUFA derivatives are selected from cyclopropanated rumenic acid, cyclopropanated alpha-elostearic acid, cyclopropanated catalpic acid, and cyclopropanated punicic acid.

Another class of PKC activators is the class of fatty acid alcohols and derivatives thereof, such as cyclopropanated PUFA and MUFA fatty alcohols. It is thought that these alcohols activate PKC by binding to the PS site. These alcohols can be derived from different classes of fatty acids.

In at least one embodiment, the PKC-activating fatty alcohols are derived from omega-3 PUFAs, omega-6 PUFAs, omega-9 PUFAs, and MUFAs, especially the fatty acids noted above. In at least one embodiment, the fatty alcohol is selected from cyclopropanated linolenyl alcohol (CP3 form shown above), cyclopropanated linoleyl alcohol (CP2 form shown above), cyclopropanated elaidic alcohol (shown above), cyclopropanated DCPLA alcohol, and cyclopropanated oleyl alcohol.

Another class of PKC activators includes fatty acid esters and derivatives thereof, such as cyclopropanated PUFA and MUFA fatty esters. In at least one embodiment, the cyclopropanated fatty esters are derived from omega-3 PUFAs, omega-6 PUFAs, omega-9 PUFAs, MUFAs, omega-5 PUFAs, and omega-7 PUFAs. These compounds are thought to activate PKC through binding on the PS site. One advantage of such esters is that they are generally considered to be more stable that their free acid counterparts.

In some embodiments, the PKC-activating fatty acid ester has the following structure:

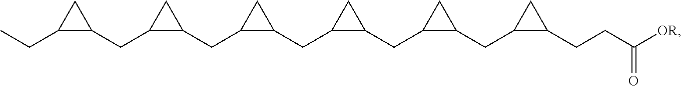

wherein R is a linear or branched alkyl group typically containing 1-6 carbon atoms, more typically 1-4, 1-3, or 1-2 carbon atoms. In some embodiments. R is methyl, ethyl, n-propyl, or isopropyl.

In one embodiment, the PKC-activating fatty acid esters derived from omega-3 PUFAs are selected from cyclopropanated eicosapentaenoic acid methyl ester (CP5 form shown below)

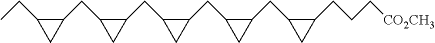 [SMILES: CCC1CC1CC1CC1CC1CC1CC1CC1CC1CC1CCCC(=O)OC]

and cyclopropanated linolenic acid methyl ester (CP3 form shown below).

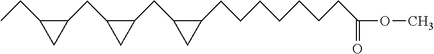 [SMILES: CCC1CC1CC1CC1CC1CC1CCCCCCCC(=O)OC]

In another embodiment, the omega-3 PUFA esters are selected from esters of DHA-CP6 and aliphatic and aromatic alcohols. In at least one embodiment, the ester is cyclopropanated docosahexaenoic acid methyl ester (CP6 form shown below).

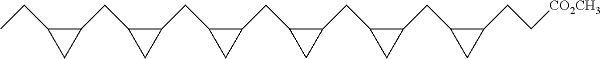 [SMILES: CCC1CC1CC1CC1CC1CC1CC1CC1CC1CC1CC1CC1CCC(=O)OC]

In one embodiment, PKC-activating fatty esters derived from omega-6 PUFAs are selected from cyclopropanated arachidonic acid methyl ester (CP4 form shown below).

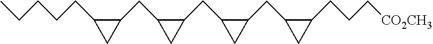 [SMILES: CCCCCC1CC1CC1CC1CC1CC1CC1CC1CCCC(=O)OC]

cyclopropanated vernolic acid methyl ester (CP1 form shown below), and

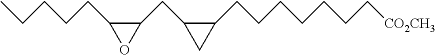 [SMILES: CCCCCC1OC1CC1CC1CCCCCCCC(=O)OC]

vernolic acid methyl ester (shown below).

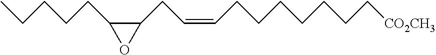 [SMILES: CCCCCC1OC1C/C=C\CCCCCCCC(=O)OC]

In particular embodiments, the PKC activating compound is an ester derivative of DCPLA (CP6-linoleic acid). In one embodiment, the ester of DCPLA is an alkyl ester. The alkyl group of the DCPLA alkyl esters may be linear, branched, and/or cyclic. The alkyl groups may be saturated or unsaturated. When the alkyl group is an unsaturated cyclic alkyl group, the cyclic alkyl group may be aromatic. The alkyl group may be selected from, for example, methyl, ethyl, propyl (e.g., isopropyl), and butyl (e.g., tert-butyl) esters. DCPLA in the methyl ester form (“DCPLA-ME”) is shown below

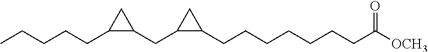 [SMILES: CCCCCC1CC1CC1CC1CCCCCCCC(=O)OC]

In another embodiment, the esters of DCPLA are derived from a benzyl alcohol (unsubstituted benzyl alcohol ester shown below). In yet another embodiment, the esters of DCPLA are derived from aromatic alcohols such as phenols used as antioxidants and natural phenols with pro-learning ability. Some specific examples include estradiol, butylated hydroxytoluene, resveratrol, polyhydroxylated aromatic compounds, and curcumin.

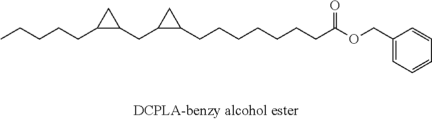 [SMILES: CCCCCC1CC1CC1CC1CCCCCCCC(=O)OCc1ccccc1]

Another class of PKC activators includes fatty esters derived from cyclopropanated MUFAs. In an embodiment, the cyclopropanated MUFA ester is selected from cyclopropanated elaidic acid methyl ester (shown below).

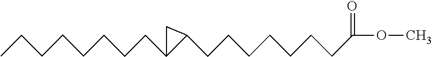 [SMILES: CCCCCCCCC1CC1CCCCCCCC(=O)OC]

and cyclopropanated oleic acid methyl ester (shown below).

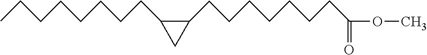 [SMILES: CCCCCCCCC1CC1CCCCCCCC(=O)OC]

Another class of PKC activators includes sulfates and phosphates derived from PUFAs, MUFAs, and their derivatives. In an embodiment, the sulfate is selected from DCPLA sulfate and DHA sulfate (CP6 form shown below).

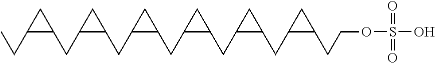 [SMILES: CCC1CC1CC1CC1CC1CC1CC1CC1CC1CC1CC1CC1CCOS(=O)(=O)O]

In one embodiment, the phosphate is selected from DCPLA phosphate and DHA phosphate (CP6 form shown below).

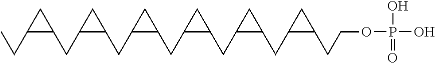 [SMILES: CCC1CC1CC1CC1CC1CC1CC1CC1CC1CC1CC1CC1CCOP(=O)(O)O]

In some embodiments, the therapeutic agent (e.g., bryostatin, bryolog or PUFA or derivative thereof) is administered in combination with one or more other substances (i.e., co-drugs, typically, non-PKC activating) known to reduce neurological pain or inflammation or to help with an associated disorder (e.g., diabetes or MS). The one or more other substances may also favorably or synergistically augment the efficacy of the therapeutic agent in treating neurological inflammation or an associated disorder. The one or more co-drugs may be administered separately (e.g., same or different days or weeks) but in tandem with the administration of the therapeutic agent, or the one or more co-drugs may be included within the same pharmaceutical formulation as the therapeutic agent, thereby being administered to the subject at the same time within the same dosage form. The co-drug may be, for example, a hormone (e.g., melatonin), antibiotic (e.g., minocycline), steroid or corticosteroid (e.g., corticotrophin, cortisone, or methylprednisolone), cytokine in the interferon family (e.g., interferon beta-la or interferon beta 1-alpha), or statin (e.g., simvastatin).

The therapeutic agent according to the present disclosure may be administered to a patient/subject in need thereof by conventional methods, such as oral, parenteral, transmucosal, intranasal, inhalation, or transdermal administration. Parenteral administration includes intravenous, intra-arteriolar, intramuscular, intradermal, subcutaneous, intraperitoneal, intraventricular, intrathecal, ICV, intracisternal injections or infusions and intracranial administration. A suitable route of administration may be chosen to permit crossing the blood-brain barrier. See e.g., J. Lipid Res. (2001) vol. 42, pp. 678-685, incorporated by reference herein.

The therapeutic agents can be compounded into a pharmaceutical composition suitable for administration to a subject using general principles of pharmaceutical compounding. In one aspect, the pharmaceutically acceptable formulation comprises a therapeutic agent (e.g., bryostatin, bryolog, or PUFA or derivative thereof) and a pharmaceutically acceptable carrier. One or more co-drugs, as described above, may or may not also be included in the formulation.

The formulations of the compositions described herein may be prepared by any suitable method known in the art. In general, such preparatory methods include bringing at least one of the therapeutic agents into association with a carrier. If necessary or desirable, the resultant product can be shaped or packaged into a desired single- or multi-dose unit.

As discussed herein, carriers include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other additional ingredients that may be included in the compositions of the disclosure are generally known in the art and may be described, for example, in Remington's Pharmaceutical Sciences, Genaro, ed., Mack Publishing Co., Easton, Pa., 1985, and Remington's Pharmaceutical Sciences, 20^{th} Ed., Mack Publishing Co. 2000, both incorporated by reference herein.

In at least one embodiment, the carrier is an aqueous or hydrophilic carrier. In a further embodiment, the carrier may be water, saline, or dimethylsulfoxide. In another embodiment, the carrier is a hydrophobic carrier. Exemplary hydrophobic carriers include, for example, inclusion complexes, dispersions (such as micelles, microemulsions, and emulsions), and liposomes. See, e.g., Remington's: The Science and Practice of Pharmacy 20th ed., ed. Gennaro, Lippincott: Philadelphia, PA 2003, incorporated by reference herein. In addition, other compounds may be included either in the hydrophobic carrier or the solution, e.g., to stabilize the formulation.

In some embodiments, the compositions described herein may be formulated into oral dosage forms. For oral administration, the composition may be in the form of a tablet or capsule prepared by conventional means with, for example, carriers such as binding agents (e.g., pregelatinized maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets may be coated by methods generally known in the art.

In another embodiment, the compositions herein are formulated into a liquid preparation. Such preparations may be in the form of, for example, solutions, syrups or suspensions, or they may be presented as a dry product for constitution with water or other suitable vehicle before use. Such liquid preparations may be prepared by conventional means using pharmaceutically acceptable carriers, such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl p-hydroxybenzoates, or sorbic acid). The preparations may also comprise buffer salts, flavoring, coloring, and sweetening agents as appropriate. In some embodiments, the liquid preparation is specifically designed for oral administration.

In another embodiment of the present disclosure, the compositions herein may be formulated for parenteral administration such as bolus injection or continuous infusion. Formulations for injection may be presented in unit dosage form, e.g., in ampoules, or in multi-dose containers, with an added preservative. The composition may be in the form of a suspension, solution, dispersion, or emulsion in oily or aqueous vehicles, and may contain a formulary agent, such as a suspending, stabilizing, and/or dispersing agent.

In another embodiment, the compositions herein may be formulated as depot preparations. Such formulations may be administered by implantation (for example, subcutaneously or intramuscularly) or by intramuscular injection. For example, the compositions may be formulated with a suitable polymeric or hydrophobic material (for example, as an emulsion in an acceptable oil) or ion exchange resin, or as a sparingly soluble derivative, for example, as a sparingly soluble salt.

In another embodiment, at least one therapeutic agent or combination thereof is delivered in a vesicle, such as a micelle, liposome, or an artificial low-density lipoprotein (LDL) particle. See, e.g., U.S. Pat. No. 7,682,627, the contents of which are herein incorporated by reference.

In some embodiments, at least one therapeutic agent or combination thereof may be present in the pharmaceutical composition in an amount ranging from about 0.01% to about 100%, from about 0.1% to about 90%, from about 0.1% to about 60%, from about 0.1% to about 30% by weight, or from about 1% to about 10% by weight of the final formulation. In another embodiment, at least one therapeutic agent or combination thereof may be present in the composition in an amount ranging from about 0.01% to about 100%, from about 0.1% to about 95%, from about 1% to about 90%, from about 5% to about 85%, from about 10% to about 80%, and from about 25% to about 75%.

The present disclosure further relates to kits that may be utilized for administering to a subject a therapeutic agent according to the present disclosure. The kits may comprise devices for storage and/or administration. For example, the kits may comprise syringe(s), needle(s), needle-less injection device(s), sterile pad(s), swab(s), vial(s), ampoule(s), cartridge(s), bottle(s), and the like. The storage and/or administration devices may be graduated to allow, for example, measuring volumes. In at least one embodiment, the kit comprises at least one therapeutic agent in a container separate from other components in the system.

The kits may also comprise one or more anesthetics, such as local anesthetics. In at least one embodiment, the anesthetics are in a ready-to-use formulation, for example, an injectable formulation (optionally in one or more pre-loaded syringes), or a formulation that may be applied topically. Topical formulations of anesthetics may be in the form of an anesthetic applied to a pad, swab, towelette, disposable napkin, cloth, patch, bandage, gauze, cotton ball, Q-tip™, ointment, cream, gel, paste, liquid, or any other topically applied formulation. Anesthetics for use with the present disclosure may include, but are not limited to lidocaine, marcaine, cocaine, and xylocaine.

The kits may also contain instructions relating to the use of at least one therapeutic agent or a combination thereof. In another embodiment, the kit may contain instructions relating to procedures for mixing, diluting, or preparing formulations of at least one therapeutic agent or a combination thereof. The instructions may also contain directions for properly diluting a formulation of at least one therapeutic agent or a combination thereof in order to obtain a desired pH or range of pHs and/or a desired specific activity and/or protein concentration after mixing but prior to administration. The instructions may also contain dosing information. The instructions may also contain material directed to methods for selecting subjects for treatment with at least one therapeutic agent or a combination thereof.

The therapeutic agent can be formulated, alone in suitable dosage unit formulations containing conventional non-toxic pharmaceutically acceptable carriers, adjuvants and vehicles appropriate for each route of administration. Pharmaceutical compositions may further comprise other therapeutically active compounds which are approved for the treatment of neurodegenerative diseases or to reduce the risk of developing a neurodegenerative disorder.

All of the references, patents and printed publications mentioned in the instant disclosure are hereby incorporated by reference in their entirety into this application.

The following examples are provided by way of illustration to further describe certain preferred embodiments of the invention, and are not intended to be limiting of the present disclosure.

### Examples

In vitro or in vivo studies may be performed using a bryostatin (e.g., bryostatin-1) or analogue thereof, such as a bryolog, or PUFA or derivative thereof. An experimental spinal cord injury mouse model, in particular, may be used for modeling nerve damage. By known methods, spinal cord injury can be first initiated (induced) or simulated in the mice. Some of the mice are then administered the bryostatin, bryolog compound, or PUFA or derivative thereof (therapeutic compound) either before, during, or after initiation to determine the effects of the compound on the spinal cord injury. When administering the therapeutic compound after initiation of spinal cord injury, the therapeutic compound may result in stabilization or even reversal of the spinal cord injury.

Groups of mice may be formed and housed in an approved research animal facility, with water given ad libitum. A first study involves three groups of mice with animals in each group dosed weekly for 1, 2, 3, or 6 consecutive weeks. Each group has its own control group containing the same number of mice. For example, mice in the first, second and third groups may receive an intravenous (i.v.) injection of 10 μg/m², 15 μg/m², and 25 μg/m² dose of bryostatin, bryolog compound, or PUFA or derivative thereof respectively. For each dose, mice in that group may receive a single injection of a bryostatin, bryolog compound, or PUFA or derivative thereof weekly for a predetermined number of consecutive weeks. Following dosing, mice are generally sacrificed and the spinal cord tissue of each animal may be collected for further analysis.

The effects of different types of intermittent dosing regimens may also be investigated. For example, in some cases, mice are dosed weekly with a bryostatin, bryolog compound, or PUFA or derivative thereof at about 25 μg/m² for three consecutive weeks, followed by cessation of drug administration for three consecutive weeks, and then a second round of dosing at about 25 μg/m² for an additional three consecutive weeks (that is, a “3 on/3 off/3 on” dosing regimen). In other embodiments, mice are dosed at about 25 μg/m² at a “1 on/1 off” regimen for a total of nine weeks (e.g., one dose of a bryostatin, bryolog compound, or PUFA or derivative thereof on weeks 1, 3, 5, 7, and 9, with no dosing in weeks 2, 4, 6, and 8). In other embodiments, mice are dosed at about 25 μg/m² for another regimen starting with “2 on/1 off” immediately followed by alternating “1 on/1 off” until reaching the ninth total week (i.e., one dose of bryostatin, bryolog compound, or PUFA or derivative thereof on weeks 1, 2, 4, 6, 8, with no dosing in weeks 3, 5, 7, and 9). Increasing the duration of the rest intervals (i.e., “off” intervals) to three weeks may significantly reduce PKC downregulation. That is, the “3 on/3 off” dosing regimen may increase brain PKC-ε levels in mice over the other regimens, thus resulting in particularly beneficial results. In another intermittent dosing regimen, mice are administered a bryostatin, bryolog compound, or PUFA or derivative thereof in a dosage of about 15, 24, or 48 μg/m² once per week for an initial one or two weeks, followed by no administration of a bryostatin, bryolog compound, or PUFA or derivative thereof (or other PKC activator) for one, two, or three weeks, followed by administration of a bryostatin, bryolog compound, or PUFA or derivative thereof in a dosage of about 12, 20, or 40 μg/m², respectively, once per week consecutively or on alternate weeks for one, two, three, four, five, six, seven, eight, nine, ten, eleven, or twelve weeks.

Brain BDNF in mice may reach its highest level after three consecutive weekly doses of a bryostatin, PUFA, derivative thereof, or other PKC activating compound at about 25 μg/m² and remain elevated after three additional consecutive weeks of no dosing, followed by three more consecutive weekly doses at about 25 μg/m². Since BDNF is a peptide that induces synaptogenesis (i.e., the formation of new synapses), a “3 on/3 off” regimen may maximize synaptogenesis and minimize PKC downregulation. Maximization of synaptogenesis and minimization of PKC downregulation is also provided by the above described intermittent dosing regimen in which mice are administered a bryostatin, bryolog compound, or PUFA or derivative thereof in a dosage of about 15, 24, or 48 μg/m² once per week for an initial one or two weeks, followed by no administration of a bryostatin, bryolog compound, or PUFA or derivative thereof (or other PKC activator) for one, two, or three weeks, followed by administration of a bryostatin, bryolog compound, or PUFA or derivative thereof in a dosage of about 12, 20, or 40 μg/m², respectively, once per week consecutively or on alternate weeks for one, two, three, four, five, six, seven, eight, nine, ten, eleven, or twelve weeks.

Further evaluation may be performed on a bryostatin, bryolog compound, or PUFA or derivative thereof crossing the blood-brain-barrier (BBB) and the steady state levels of bryostatin, bryolog compound, or PUFA or derivative thereof in the brain and plasma of mice. In some embodiments, a bryostatin, bryolog compound, or PUFA or derivative thereof administered intravenously crosses the BBB. In that case, the concentration of bryostatin, bryolog compound, or PUFA or derivative thereof in the mouse brain may be less than its concentration in plasma. However, the concentration in the brain may be no less than two-fold lower than the plasma concentrations for comparable doses under steady-state conditions.

A weekly dosing regimen of a single injection of bryostatin, bryolog compound, or PUFA or derivative thereof at a dose of about 25 μg/m² for three consecutive weeks may be less effective at increasing bryostatin, bryolog, or PUFA concentration in the mouse brain than a “1 on/1 off” or a “2 on/1 off” administration of the 25 μg/m² dose. In contrast, plasma concentrations of the bryostatin, bryolog compound, or PUFA or derivative thereof may be greater when the drug is administered as a single injection for three consecutive weeks. Blood plasma concentrations of the bryostatin, bryolog compound, or PUFA or derivative thereof may be less in mice receiving a 25 μg/m² dose as a “1 on/1 off” or a “2 on/1 off” administration. Without being bound to a specific theory, it may be hypothesized that the intermittent dosing regimen, such as any of those described above, facilitates the transport of bryostatin or bryolog compound or PUFA or derivative thereof across the BBB.

While there have been shown and described what are at present considered the preferred embodiments of the invention, those skilled in the art may make various changes and modifications which remain within the scope of the invention defined by the appended claims.

1. A method for treating nerve damage in a subject in need thereof, the method comprising administering to the subject a pharmaceutically effective amount of a therapeutic agent on a weekly dosage regimen to result in at least partial repair or regeneration of damaged nerves in the subject, wherein the weekly dosage regimen comprises a dosage of about 10 to about 60 μg/m² once a week for at least two weeks, wherein the at least two weeks of dosing are done consecutively or intermittently, wherein the therapeutic agent is selected from bryostatin compounds, polyunsaturated fatty acids (PUFAs), and derivatives thereof.

2. The method of claim 1, wherein the weekly dosage regimen comprises a dosage of about 10 to about 60 μg/m² once a week for at least three weeks, wherein the at least three weeks of dosing are done consecutively or intermittently.

3. The method of claim 1, wherein the weekly dosage regimen comprises a dosage of about 10 to about 60 μg/m² once a week for at least four weeks, wherein the at least four weeks of dosing are done consecutively or intermittently.

4. The method of claim 1, wherein the weekly dosage regimen comprises a dosage of about 10 to about 60 μg/m² once a week for at least two consecutive weeks followed by a dosage of about 10 to about 60 μg/m² once a week for at least two weeks done intermittently.

5. The method of claim 1, wherein the dosage is, in each instance, independently about 10 to about 50 mg/m².

6. The method of claim 1, wherein the dosage is, in each instance, independently about 10 to about 40 mg/m².

7. The method of claim 1, wherein the weekly dosage regimen is done intermittently as follows: (i) a first period in which the subject is administered the therapeutic agent in a dosage of about 10 to about 60 μg/m² once a week for 1-3 weeks consecutively, (ii) a second period of 1-3 weeks in which the subject is not administered the therapeutic agent, and (iii) a third period in which the subject is administered the therapeutic agentin a dosage of about 10 to about 60 μg/m² once a week for at least one week done consecutively or intermittently.

8. The method of claim 1, wherein the weekly dosage regimen is done intermittently as follows: (i) a first period in which the subject is administered the therapeutic agent in a dosage of about 10 to about 60 mg/m² once a week for 2-3 weeks consecutively, (ii) a second period of 1-3 weeks in which the subject is not administered the therapeutic agent, and (iii) a third period in which the subject is administered the therapeutic agentin a dosage of about 10 to about 60 μg/m² once a week for at least two weeks done consecutively or intermittently.

9. The method of claim 1, wherein the weekly dosage regimen is done intermittently as follows: (i) a first period in which the subject is administered the therapeutic agent in a dosage of about 10 to about 60 mg/m² once a week for 2-3 weeks consecutively, (ii) a second period of 1-3 weeks in which the subject is not administered the therapeutic agent, and (iii) a third period in which the subject is administered the therapeutic agent in a dosage of about 10 to about 60 μg/m² once a week for at least three weeks done consecutively or intermittently.

10. The method of claim 1, wherein the weekly dosage regimen is done intermittently as follows: (i) a first period in which the subject is administered the therapeutic agent in a loading dosage of 13-20 μg/m² once a week for 1-3 weeks consecutively, (ii) a second period of 1-3 weeks in which the subject is not administered the therapeutic agent, and (iii) a third period in which the subject is administered the therapeutic agent in a dosage of about 10-17 μg/m² once a week for at least two weeks done consecutively or intermittently, wherein the loading dosage is at least 3 μg/m² higher than the dosage in the third period.

11. The method of claim 10, wherein the weekly dosage regimen is done intermittently as follows: (i) a first period in which the subject is administered the therapeutic agent in a loading dosage of about 15 μg/m² once a week for 1-3 weeks consecutively, (ii) a second period of 1-3 weeks in which the subject is not administered the therapeutic agent, and (iii) a third period in which the subject is administered the therapeutic agent in a dosage of about 12 μg/m² once a week for at least two weeks done consecutively or intermittently.

12. The method of claim 1, wherein the weekly dosage regimen is done intermittently as follows: (i) a first period in which the subject is administered the therapeutic agent in a loading dosage of 20-30 μg/m² once a week for 1-3 weeks consecutively, (ii) a second period of 1-3 weeks in which the subject is not administered the therapeutic agent, and (iii) a third period in which the subject is administered the therapeutic agent in a dosage of about 15-27 μg/m² once a week for at least two weeks done consecutively or intermittently, wherein the loading dosage is at least 3 μg/m² higher than the dosage in the third period.

13. The method of claim 12, wherein the weekly dosage regimen is done intermittently as follows: (i) a first period in which the subject is administered the therapeutic agent in a loading dosage of about 24 μg/m² once a week for 1-3 weeks consecutively, (ii) a second period of 1-3 weeks in which the subject is not administered the therapeutic agent, and (iii) a third period in which the subject is administered the therapeutic agent in a dosage of about 20 μg/m² once a week for at least two weeks done consecutively or intermittently.

14. The method of claim 1, wherein the weekly dosage regimen is done intermittently as follows: (i) a first period in which the subject is administered the therapeutic agent in a loading dosage of 30-60 μg/m² once a week for 1-3 weeks consecutively, (ii) a second period of 1-3 weeks in which the subject is not administered the therapeutic agent, and (iii) a third period in which the subject is administered the therapeutic agent in a dosage of about 27-57 μg/m² once a week for at least two weeks done consecutively or intermittently, wherein the loading dosage is at least 3 μg/m² higher than the dosage in the third period.

15. The method of claim 14, wherein the weekly dosage regimen is done intermittently as follows: (i) a first period in which the subject is administered the therapeutic agent in a loading dosage of about 48 μg/m² once a week for 1-3 weeks consecutively, (ii) a second period of 1-3 weeks in which the subject is not administered the therapeutic agent, and (iii) a third period in which the subject is administered the therapeutic agent in a dosage of about 40 μg/m² once a week for at least two weeks done consecutively or intermittently.

16. The method of claim 1, wherein the subject is administered a bryostatin compound as the therapeutic agent.

17. The method of claim 9, wherein the bryostatin compound is bryostatin-1.

18. The method of claim 1, wherein the subject is administered a bryolog compound.

19. The method of claim 18, wherein the bryolog compound has any of the following structures: 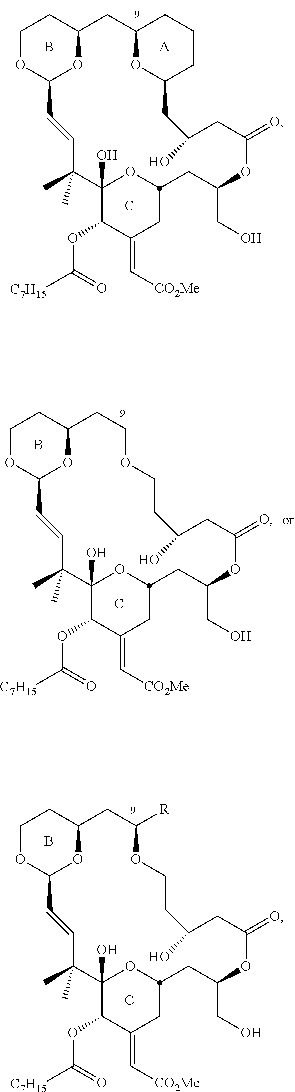
wherein R is selected from t-butyl, phenyl, and (CH₂)₃-p-Br-phenyl.

20. The method of claim 1, wherein the subject is administered a PUFA compound or derivative thereof as the therapeutic agent.

21. The method of claim 20, wherein the PUFA compound is a cyclopropanated PUFA.

22. The method of claim 20, wherein the PUFA compound is a polyunsaturated fatty acid ester.

23. The method of claim 22, wherein the polyunsaturated fatty acid ester has the following structure: 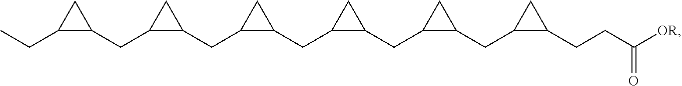
wherein R is a linear or branched alkyl group containing 1-6 carbon atoms.

24. The method of claim 1, wherein the therapeutic agent is administered intravenously.

25. The method of claim 1, wherein the therapeutic agent is administered as an oral dosage form.

26. The method of claim 1, wherein the subject has spinal cord injury.

27. The method of claim 1, wherein the subject has cochlear nerve damage.

28. The method of claim 1, wherein the subject has nerve damage to direct peripheral nerves.
